(19) United States
(12) Patent Application Publication
(10) Pub. No.: US 2026/0256895 A1
(43) Pub. Date: Sep. 3, 2026

(54) IMPROVED DOWNREGULATION OF HISTONE CYTOTOXICITY BY COMPLEX OF NEGATIVELY CHARGED POLYSACCHARIDE AND PROTEASE

(71) Applicants: UNIVERSITEIT MAASTRICHT, MAASTRICHT (NL); ACADEMISCH ZIEKENHUIS MAASTRICHT, MAASTRICHT (NL)

(72) Inventors: Gerardus Anna Franciscus NICOLAES, MAASTRICHT (NL); Christiaan Peter Maria REUTELINGSPERGER, MAASTRICHT (NL); Joram Bernd HUCKRIEDE, MAASTRICHT (NL)

(21) Appl. No.: 18/880,021

(22) PCT Filed: Jul. 3, 2023
(86) PCT No.: PCT/EP2023/068246

§ 371 Date: Dec. 30, 2024

(30) Foreign Application Priority Data
Jul. 1, 2022 (EP) 22182526.8

(51) Int. Cl. A61K 38/48; A61K 47/55; A61K 47/61

(52) U.S. Cl. CPC A61K 38/486; A61K 38/4866; A61K 47/55; A61K 47/61

(57) ABSTRACT

Described herein is a complex of a negatively charged polysaccharide and a protein having serine protease activity. The negatively charged polypeptide can for example be heparin or non-anticoagulant heparin. The serine protease can for example be an elastase or activated protein C. The complex can be used in the treatment, prevention or amelioration of an immunothrombosis related disease or disorder.

### FIELD OF THE INVENTION

The invention relates to a complex of negatively charged polysaccharide and a protein having protease activity. The inventors show that extracellular histones can be degraded using such complex. Such complex has particular use in treating, preventing or ameliorating extracellular histone-mediated disease or disorders or a disease or disorder where immunothrombosis is problematic, such as for example sepsis and Covid-19. Non-limiting exemplary complexes according to the invention are complexes of heparin or non-anticoagulant heparin with either an elastase or activated protein C.

### BACKGROUND OF THE INVENTION

Extracellular histones (eHs) can be found in circulation of patients suffering from extracellular histone-mediated related diseases or disorders such as acute inflammatory diseases like sepsis and COVID-19, but have also been demonstrated to play a role in trauma, stroke or pancreatitis. The circulating eHs can be proteolytically cleaved. Proteolytic cleavage of eHs destroys their cytotoxic activity. Patients whose circulating eHs have not been cleaved have a significantly higher risk of aggravating morbidity and mortality.

Over the past years it has become clear that eHs play a major role in driving diseases such as systemic inflammatory response syndrome, sepsis and COVID-19. Recently it was shown that eHs significantly promote progression of prostate cancer via NF-κB pathway-mediated inflammatory responses. Hence, neutralising eHs is a promising therapy to treat cancer patients, especially patients who are treated with chemotherapeutics and irradiation, which increase eHs dramatically. Also in organ preservation/transplantation eHs worsen the outcome of transplantation, while in atherosclerosis, eHs-mediated lysis of smooth muscle cells (SMCs) was found to trigger arterial tissue damage and inflammation. Pharmacological neutralization of eHs is therefore appreciated as an important strategy to treat these type of diseases. Heparin has been suggested as a treatment strategy for several indications where eHs play a role, however the dual action of heparin, having an anti-inflammatory and anticoagulant action may provide undesired effects. Therefore low anticoagulant heparin has been suggested as an alternative. Low anticoagulant heparin, which neutralizes eHs by electrostatic interaction, is currently under investigation for treatment of patients with sepsis and COVID-19. However, low anticoagulant heparin may still result in undesired effects in patients like undesirable elevated activated partial thromboplastine time (aPTT), therefore formulations with improved effect or less side effects are desirable. The invention as described in the appended claims aims to overcome these problems, among others.

EP0326014A1 describes the use of a composition comprising heparin and APC as an improved anticoagulant. Pejler et al. (Biofactors Volume 35, Number 1, January/February 2009, Pages 61-68) relates to the role of serglycin in promoting the storage and in regulating the activities of a number of proteases expressed in hematopoietic cell types, most notably various mast cell proteases. It incidentally describes that interactions of Mast Cell proteases and heparin are likely to occur in vivo. Kummarapurugu et al. (J. Biol. Chem. (2018) 293(32) 12480-12490) describes that neutrophil elastase is inhibited by heparin in cystic fibrosis patients. Wildhagen et al. (BLOOD, 13 February 2014•VOLUME 123, NUMBER 7 pages 1098-1101) teaches the use of heparin in sepsis by reducing clotting. Kowalska et al. (Arterioscler Thromb Vase Biol. 2014; 34:120-126.) teaches that heparin modulates activated protein C levels which may be beneficial in sepsis.

### SUMMARY OF THE INVENTION

In a first aspect the invention relates to a complex of a negatively charged polysaccharide and a protein having protease activity.

In a second aspect the invention relates to a pharmaceutical composition comprising the complex according to the first aspect of the invention and a pharmaceutically acceptable carrier.

In a third aspect the invention relates to the complex according to the first aspect of the invention or the pharmaceutical composition according to the second aspect of the invention for use as a medicament.

In a fourth aspect the invention relates to the complex according to the first aspect of the invention or the pharmaceutical composition according to the second aspect of the invention for use in the treatment, prevention or amelioration of an extracellular histone-mediated disease or disorder.

### FIGURE LEGENDS

FIG. 1: Detection of Histone cleavage with Western blot of Histones H2B, H3, and H4.

FIG. 2: Time course of Histone degradation by NE and NE complexed with UFH.

FIG. 3: Dose response of heparins UFH and M6229 on the viability of EA.hy926 cells that were incubated with Histone H3. Complexation with NE enhances the cytoprotective effects of heparins.

FIG. 4: Time course of Histone H3 degradation by APC and APC complexed with UFH.

FIG. 5: Dose response of UFH on the viability of EA.hy926 cells that were incubated with Histone H3. Complexation with APC enhances the cytoprotective effects of heparins.

### DEFINITIONS

For purposes of the present invention, the following terms are defined below.

As used herein, the singular form terms “A,” “an,” and “the” include plural referents unless the content clearly dictates otherwise. Thus, for example, reference to “a cell” includes a combination of two or more cells, and the like.

As used herein, the term “and/or” refers to a situation wherein one or more of the stated cases may occur, alone or in combination with at least one of the stated cases, up to with all of the stated cases.

As used herein, the term “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc. As used herein, the term “at most” a particular value means that particular value or less. For example, “at most 5” is understood to be the same as “5 or less” i.e., 5, 4, 3, . . . −10, −11, etc.

As used herein, the word “comprise” or variations thereof such as “comprises” or “comprising” will be understood to include a stated element, integer or step, or group of elements, integers or steps, but not to exclude any other element, integer or steps, or groups of elements, integers or steps. The verb “comprising” includes the verbs “essentially consisting of” and “consisting of”.

As used herein, the term “conventional techniques” refers to a situation wherein the methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al., Molecular Cloning. A Laboratory Manual, 2nd Edition, Cold Spring Harbor Laboratory Press, Cold Spring Harbor, N. Y., 1989; Ausubel et al., Current Protocols in Molecular Biology, John Wiley & Sons, New York, 1987 and periodic updates; and the series Methods in Enzymology, Academic Press, San Diego.

As used herein, the term “identity” refers to a measure of the identity of nucleotide sequences or amino acid sequences. In general, the sequences are aligned so that the highest order match is obtained. “Identity” per se has an art-recognized meaning and can be calculated using published techniques. See, e.g.: (Computational Molecular Biology, Lesk, A. M., ED., Oxford University Press, New York, 1988; Biocomputing: Informatics And Genome Projects, Smith, D. W., ED., Academic Press, New York, 1993; Computer Analysis Of Sequence Data, Part I, Griffin, A. M., And Griffin, H. G., EDS., Humana Press, New Jersey, 1994; Sequence Analysis In Molecular Biology, Von Heinje, G., Academic Press, 1987; and Sequence Analysis Primer; Gribskov, M. and Devereux, J., eds., M Stockton Press, New York, 1991). While there exist a number of methods to measure identity between two nucleotide sequences or amino acid sequences, the term “identity” is well known to skilled artisans (Carillo, H., and Lipton, D., SIAM J. Applied Math (1988) 48:1073). Methods commonly employed to determine identity or similarity between two sequences include, but are not limited to, those disclosed in Guide To Huge Computers, Martin J. Bishop, ed., Academic Press, San Diego, 1994, and Carillo, H., and Lipton, D., Siam J. Applied Math (1988) 48:1073. Methods to determine identity and similarity are codified in computer programs. Preferred computer program methods to determine identity and similarity between two sequences include, but are not limited to, GCS program package (Devereux, J., et al., Nucleic Acids Research (1984) 12(1):387), BLASTP, BLASTN, FASTA (Atschul, S. F. et al., J. Molec. Biol. (1990) 215:403).

As an illustration, by a polynucleotide having a nucleotide sequence having at least, for example, 95% “identity” to a reference nucleotide sequence encoding a polypeptide of a certain sequence, it is intended that the nucleotide sequence of the polynucleotide is identical to the reference sequence except that the polynucleotide sequence may include up to five point mutations per each 100 nucleotides of the reference amino acid sequence. In other words, to obtain a polynucleotide having a nucleotide sequence at least 95% identical to a reference nucleotide sequence, up to 5% of the nucleotides in the reference sequence may be deleted and/or substituted with another nucleotide, and/or a number of nucleotides up to 5% of the total nucleotides in the reference sequence may be inserted into the reference sequence. These mutations of the reference sequence may occur at the 5′ or 3′ terminal positions of the reference nucleotide sequence, or anywhere between those terminal positions, interspersed either individually among nucleotides in the reference sequence or in one or more contiguous groups within the reference sequence. Similarly, by a polypeptide having an amino acid sequence having at least, for example, 95% “identity” to a reference amino acid sequence of SEQ ID NO: X is intended that the amino acid sequence of the polypeptide is identical to the reference sequence except that the amino acid sequence may include up to five amino acid alterations per each 100 amino acids of the reference amino acid of SEQ ID NO: X. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As used herein, the term “in vitro” refers to experimentation or measurements conducted using components of an organism that have been isolated from their natural conditions.

As used herein, the term “ex vivo” refers to experimentation or measurements done in or on tissue from an organism in an external environment with minimal alteration of natural condition.

As used herein, the term “nucleic acid”, “nucleic acid molecule” and “polynucleotide” is intended to include DNA molecules and RNA molecules, as well as locked nucleic acid (LNA), bridged nucleic acid (BNA), morpholino or peptide nucleic acid (PNA). A nucleic acid (molecule) may be any nucleic acid (molecule), it may be single-stranded or double-stranded.

As used herein, the terms “sequence” when referring to nucleotides, or “nucleic acid sequence”, “nucleotide sequence” or “polynucleotide sequence” refer to the order of nucleotides of, or within, a nucleic acid and/or polynucleotide. Within the context of the current invention a first nucleic acid sequence may be comprised within or overlap with a further nucleic acid sequence.

As used herein, the term “subject” or “individual” or “animal” or “patient” or “mammal,” used interchangeably, refer to any subject, particularly a mammalian subject, for whom diagnosis, prognosis, or therapy is desired. Mammalian subjects include humans, domestic animals, farm animals, and zoo-, sports-, or pet-animals such as dogs, cats, guinea pigs, rabbits, rats, mice, horses, cattle, cows, bears, and so on. As defined herein a subject may be alive or dead. Samples can be taken from a subject post-mortem, i.e. after death, and/or samples can be taken from a living subject.

As used herein, terms “treatment”, “treating”, “palliating”, “alleviating” or “ameliorating”, used interchangeably, refer to an approach for obtaining beneficial or desired results including, but not limited to, therapeutic benefit. By therapeutic benefit is meant eradication or amelioration or reduction (or delay) of progress of the underlying disease being treated. Also, a therapeutic benefit is achieved with the eradication or amelioration or reduction (or delay) of progress of one or more of the physiological symptoms associated with the underlying disease such that an improvement or slowing down or reduction of decline is observed in the patient, notwithstanding that the patient can still be afflicted with the underlying disease.

### DETAILED DESCRIPTION

The section headings as used herein are for organizational purposes only and are not to be construed as limiting the subject matter described.

A portion of this invention contains material that is subject to copyright protection (such as, but not limited to, diagrams, device photographs, or any other aspects of this submission for which copyright protection is or may be available in any jurisdiction). The copyright owner has no objection to the facsimile reproduction by anyone of the patent document or patent invention, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention relates, unless otherwise indicated. Other specifically defined terms are to be construed in a manner consistent with the definition as provided herein. The preferred materials and methods are described herein, although any methods and materials similar or equivalent to those described herein can be used in the practice for testing of the present invention. Unless otherwise defined, all technical and scientific terms used herein have the same meaning as commonly understood by a person of ordinary skill in the art.

The present invention is based on the observation that neutrophil elastase (NE), which is released by activated neutrophils that participate in acute inflammatory responses, can cleave eHs. The inventors have further demonstrated that the cleavage of eHs by NE is dramatically enhanced by heparins including the low anticoagulant heparins such as M6229. NE is a serine protease. In addition, it was found that another serine protease, APC, also cleaves histones and that heparins accelerate the APC-catalyzed cleavage of eHs, indicating a general principle for heparin-enhanced proteolysis of eHs by proteases.

Without being bound to theory, the mechanism of catalysis is thought to be based on the approximation principle, being that the electrostatic interaction of heparin with eH enhances the functional collision between the protease and eH. Therefore, the inventors theorized that the principle may be applied to any negatively charged polysaccharide in complex with any protein with proteolytic activity.

Therefore, the present invention describes a novel composition of matter describing a complex of a protein with protease activity and a negatively charged polysaccharide that has improved eHs neutralizing activity. In a first embodiment, the invention thus relates to a complex of a negatively charged polysaccharide and a protein having serine protease activity. These complexes are particularly useful in the treatment, prevention or amelioration of an extracellular histone-mediated disease or disorder.

The present invention improves the ability of negatively charged polysaccharides such as heparins to neutralize eHs by adding a proteolytic component (e.g. a protease such as a serine protease). This will enhance the neutralizing power of negatively charged polysaccharides and lower the required dose of negatively charged polysaccharides (e.g. heparin) to treat patients and, hence, will lower the risk that the treated patients will develop a self-enforcing cascade of histone release leading to serious tissue damage. The invention is novel as to the best of the knowledge of the inventors such complexes have not been described in the literature. The invention can also be considered as an improvement of low anticoagulant heparin wherein the presently described complex has increased applications and indications because of improved specific activity, and the lowered risk to develop a self-enforcing cascade of histone release leading to serious tissue damage.

The literature reports that heparin inactivates the serine proteases leukocyte elastase (1) and activated protein C (2). Therefore, it is surprising that the inventors herein describe that heparin stimulates the cleavage of histones by proteases, see Examples 1 and 2. Low anticoagulant heparin, which neutralizes eHs by electrostatic interaction, is currently under investigation for treatment of sepsis and COVID-19. Our invention improves the ability of heparins to inactivate eHs by adding the proteolytic component. This will lower the required dose to treat patients and will lower the risk that the treated patients will develop a self-enforcing cascade of histone release leading to serious tissue damage.

Therefore, the present invention aims to improve upon a treatment with low anticoagulant heparin by using the complex described herein. The improved effect is anticipated to reside from the following observations:
Low anticoagulant heparin neutralizes eHs by electrostatic interaction, however the complex described herein results in the degradation of eHs to non-toxic protein fragments, therefore this is anticipated to result in a more effective treatment;
Furthermore it is anticipated that the complex as described herein allows for the use of a much lower concentration of negatively charged polysaccharide (such as for example low anticoagulant heparin), at least in part because the protease cleaves the eH and can, after such action move to a next eH.

In a particularly preferred embodiment the invention further refers to a complex of a negatively charged polysaccharide and a protein having protease activity, wherein the protein having protease activity is a serine protease, a cysteine protease, a threonine protease, an aspartic protease, a glutamic protease, a metalloprotease, or an asparagine peptide lyase, and wherein the negatively charged polysaccharide is a glycosaminoglycan (GAG); and wherein the negatively charged polysaccharide and the protein having protease activity are covalently bound.

Such complexes are also useful in the treatment, prevention or amelioration of an extracellular histone-mediated disease or disorder, but have the added advantage that the protease and the polysaccharide do not dissociate when administered to a subject in need thereof and instead remain in a complex. It is anticipated that such complexes may have even better protective properties against the cytotoxic effect caused by eHs.

The skilled person is aware how such covalently bound complexes can be generated. For example, a negatively charged polysaccharide as described herein can be crosslinked to a protein having protease activity as described herein using commonly used methods. A non-limiting example is using a crosslinker such as described in Ohnishi et al. (9) using N-bromoacetylsulfanilyl chloride or N-bromoacetamidobenzoyl chloride as heterobifunctional crosslinkers. Alternatively, polysaccharides like heparin may contain a residual amino acid like serine (10) which can be used to link the polysaccharide to a protein such as a protease, for example by using an amino bond of the residual serine with the protein. The skilled person is aware that these are only exemplary ways to create a covalently linked complex as described herein, and other options are readily available.

When used herein, a protein having protease activity or a protease refers to an enzyme that catalyzes proteolysis, breaking down proteins into smaller polypeptides or single amino acids, and spurring the formation of new protein products by cleaving the peptide bonds within proteins by hydrolysis. Proteases may be divided in exopeptidases, which detach the terminal amino acids from the protein or peptide, and endopeptidases which hydrolyse internal peptide bonds. For the purpose of the invention the protease or protein having protease activity is preferably an endopeptidase. Proteases may further be subdivided based on catalytic residue as: serine proteases—using a serine alcohol, cysteine proteases—using a cysteine thiol, threonine proteases—using a threonine secondary alcohol, aspartic proteases—using an aspartate carboxylic acid, glutamic proteases—using a glutamate carboxylic acid, metalloproteases—using a metal, usually zinc, and asparagine peptide lyases—using an asparagine to perform an elimination reaction (not requiring water). Therefore in an embodiment the protein having protease activity is a serine protease, a cysteine protease, a threonine protease, an aspartic protease, a glutamic protease, a metalloprotease, or an asparagine peptide lyase. In a preferred embodiment the protein having protease activity is a serine protease.

When used herein a protein having serine protease activity refers to an enzyme capable of cleaving peptide bonds in proteins and wherein serine serves as the nucleophilic amino acid at the enzyme's active site. Preferably the protein having protease activity is a serine protease, also known as serine endopeptidase, or derivative thereof. Serine proteases can be divided based on their substrate specificity in the following groups: Trypsin-like, Chymotrypsin-like, Thrombin-like, Elastase-like and Subtilisin-like. It is envisioned that any of these groups may be used in the invention. Therefore, in an embodiment, the protein having serine protease activity is a Trypsin-like, Chymotrypsin-like, Thrombin-like, Elastase-like or Subtilisin-like serine protease. Serine proteases are classified with the EC number EC 3.4.21 as determined by the Nomenclature Committee of the International Union of Biochemistry and Molecular Biology. Therefore, the protein with serine protease activity may be selected from EC 3.4.21. 1: chymotrypsin, EC 3.4.21.2: chymotrypsin C, EC 3.4.21.3: metridin, EC 3.4.21.4: trypsin, EC 3.4.21.5: thrombin, EC 3.4.21.6: coagulation factor Xa, EC 3.4.21.7: plasmin, EC 3.4.21.9: enteropeptidase, EC 3.4.21.10: acrosin, EC 3.4.21.12: a-lytic endopeptidase, EC 3.4.21.19: glutamyl endopeptidase, EC 3.4.21.20: cathepsin G, EC 3.4.21.21: coagulation factor Vila, EC 3.4.21.22: coagulation factor IXa, EC 3.4.21.25: cucumisin, EC 3.4.21.26: prolyl oligopeptidase, EC 3.4.21.27: coagulation factor Xia, EC 3.4.21.32: brachyuran, EC 3.4.21.34: plasma kallikrein, EC 3.4.21.35: tissue kallikrein, EC 3.4.21.36: pancreatic elastase, EC 3.4.21.37: leukocyte elastase, EC 3.4.21.38: coagulation factor XIIa, EC 3.4.21.39: chymase, EC 3.4.21.41: Complement subcomponent C1r, EC 3.4.21.42: complement subcomponent C1s, EC 3.4.21.43: classical-complement-pathway C3/C5 convertase, EC 3.4.21.45: complement factor I, EC 3.4.21.46: complement factor D, EC 3.4.21.47: alternative-complement-pathway C3/C5 convertase, EC 3.4.21.48: cerevisin, EC 3.4.21.49: hypodermin C, EC 3.4.21.50: lysyl endopeptidase, EC 3.4.21.53: edopeptidase La, EC 3.4.21.54: v-renin, EC 3.4.21.55: venombin AB, EC 3.4.21.57: leucyl endopeptidase, EC 3.4.21.59: tryptase, EC 3.4.21.60: scutellarin, EC 3.4.21.61: kexin, EC 3.4.21.62: subtilisin, EC 3.4.21.63: oryzin, EC 3.4.21.64: endopeptidase K, EC 3.4.21.65: thermomycolin, EC 3.4.21.66: thermitase, EC 3.4.21.67: endopeptidase So, EC 3.4.21.68: t-plasminogen activator, EC 3.4.21.69: protein C (activated), EC 3.4.21.70: pancreatic endopeptidase E, EC 3.4.21.71: pancreatic elastase II, EC 3.4.21.72: IgA-specific serine endopeptidase, EC 3.4.21.73: u-plasminogen activator, EC 3.4.21.74: venombin A, EC 3.4.21.75: furin, EC 3.4.21.76: myeloblastin, EC 3.4.21.77: semenogelase, EC 3.4.21.78: granzyme A, EC 3.4.21.79: granzyme B, EC 3.4.21.80: streptogrisin A, EC 3.4.21.81: streptogrisin B, EC 3.4.21.82: glutamyl endopeptidase II, EC 3.4.21.83: oligopeptidase B, EC 3.4.21.84: limulus clotting factor C, EC 3.4.21.85: limulus clotting factor B, EC 3.4.21.86: limulus clotting enzyme, EC 3.4.21.88: repressor LexA, EC 3.4.21.89: signal peptidase I, EC 3.4.21.90: togavirin, EC 3.4.21.91: flavivirin, EC 3.4.21.92: endopeptidase Clp, EC 3.4.21.93: proprotein convertase 1, EC 3.4.21.94: proprotein convertase 2, EC 3.4.21.95: snake venom factor V activator, EC 3.4.21.96: lactocepin, EC 3.4.21.97: assembling, EC 3.4.21.98: hepacivirin, EC 3.4.21.99: spermosin, EC 3.4.21.100: sedolisin, EC 3.4.21.101: xanthomonalisin, EC 3.4.21.102: C-terminal processing peptidase, EC 3.4.21.103: physarolisin, EC 3.4.21.104: mannan-binding lectin-associated serine protease- 2, EC 3.4.21.105: rhomboid protease, EC 3.4.21.106: hepsin, EC 3.4.21.107: peptidase Do, EC 3.4.21.108: HtrA 2 peptidase, EC 3.4.21.109: matriptase, EC 3.4.21.110: C 5a peptidase, EC 3.4.21.111: aqualysin 1, EC 3.4.21.112: site-1 protease, EC 3.4.21.113: pestivirus NS3 polyprotein peptidase, EC 3.4.21.114: equine arterivirus serine peptidase, EC 3.4.21.115: infectious pancreatic necrosis birnavirus Vp4 peptidase, EC 3.4.21.116: SpolVB peptidase, EC 3.4.21.117: stratum corneum chymotryptic enzyme, EC 3.4.21. 118: kallikrein 8, EC 3.4.21. 119: kallikrein 13, EC 3.4.21.120: oviductin, and EC 3.4.21.121: Lys-Lys/Arg-Xaa endopeptidase.

When used herein the term protein having cysteine protease activity or cysteine protease, also known as thiol protease, refers to a hydrolase enzyme that degrades proteins. These proteases share a common catalytic mechanism that involves a nucleophilic cysteine thiol in a catalytic triad or dyad. Such enzymes are classified as EC 3.4.22 using the Enzyme Commission number classification. Therefore, the cysteine protease may be selected from: EC 3.4.22.1: cathepsin B, EC 3.4.22.2: papain, EC 3.4.22.3: ficain, EC 3.4.22.6: chymopapain, EC 3.4.22.7: asclepain, EC 3.4.22.8: clostripain, EC 3.4.22.10: streptopain, EC 3.4.22.14: actinidain, EC 3.4.22.15: cathepsin L, EC 3.4.22.16: cathepsin H, EC 3.4.22.24: Cathepsin T, EC 3.4.22.25: Glycyl endopeptidase, EC 3.4.22.26: Cancer procoagulant, EC 3.4.22.27: cathepsin S, EC 3.4.22.28: picornain 3C, EC 3.4.22.29: picornain 2A, EC 3.4.22.30: Caricain, EC 3.4.22.31: Ananain, EC 3.4.22.32: Stem bromelain, EC 3.4.22.33: Fruit bromelain, EC 3.4.22.34: Legumain, EC 3.4.22.35: Histolysain, EC 3.4.22.36: caspase-1, EC 3.4.22.37: Gingipain R, EC 3.4.22.38: Cathepsin K, EC 3.4.22.39: adenain, EC 3.4.22.40: bleomycin hydrolase, EC 3.4.22.41: cathepsin F, EC 3.4.22.42: cathepsin O, EC 3.4.22.43: cathepsin V, EC 3.4.22.44: nuclear-inclusion-a endopeptidase, EC 3.4.22.45: helper-component proteinase, EC 3.4.22.46: L-peptidase, EC 3.4.22.47: gingipain K, EC 3.4.22.48: staphopain, EC 3.4.22.49: separase, EC 3.4.22.50: V-cath endopeptidase, EC 3.4.22.51: cruzipain, EC 3.4.22.52: calpain-1, EC 3.4.22.53: calpain-2, EC 3.4.22.54: calpain-3, EC 3.4.22.55: caspase-2, EC 3.4.22.56: caspase-3, EC 3.4.22.57: caspase-4, EC 3.4.22.58: caspase-5, EC 3.4.22.59: caspase-6, EC 3.4.22.60: caspase-7, EC 3.4.22.61: caspase-8, EC 3.4.22.62: caspase-9, EC 3.4.22.63: caspase-10, EC 3.4.22.64: caspase-11, EC 3.4.22.65: peptidase 1 (mite), EC 3.4.22.66: calicivirin, EC 3.4.22.67: zingipain, EC 3.4.22.68: Ulp1 peptidase, EC 3.4.22.69: SARS coronavirus main proteinase, EC 3.4.22.70: sortase A, and EC 3.4.22.71: sortase B.

When used herein the term protein having threonine protease activity or threonine protease, are a family of proteolytic enzymes harbouring a threonine (Thr) residue within the active site. Such enzymes are classified as EC 3.4.25 using the Enzyme Commission number classification. Therefore, the threonine protease may be selected from: EC 3.4.25.1: proteasome endopeptidase complex and EC 3.4.25.2: HsIU-HsIV peptidase.

When used herein the term protein having aspartic protease activity or aspartic protease, are a catalytic type of protease enzymes that use an activated water molecule bound to one or more aspartate residues for catalysis of their peptide substrates. In general, they have two highly conserved aspartates in the active site and are optimally active at acidic pH. Such enzymes are classified as EC 3.4.23 using the Enzyme Commission number classification. Therefore, the aspartic protease may be selected from: EC 3.4.23.1: pepsin A, EC 3.4.23.2: pepsin B, EC 3.4.23.3: gastricsin, EC 3.4.23.4: chymosin, EC 3.4.23.5: cathepsin D, EC 3.4.23.12: nepenthesin, EC 3.4.23.15: renin, EC 3.4.23.16: HIV-1 retropepsin, EC 3.4.23.17: pro-opiomelanocortin converting enzyme, EC 3.4.23.18: aspergillopepsin I, EC 3.4.23.19: aspergillopepsin II, EC 3.4.23.20: penicillopepsin, EC 3.4.23.21: rhizopuspepsin, EC 3.4.23.22: endothiapepsin, EC 3.4.23.23: mucorpepsin, EC 3.4.23.24: candidapepsin, EC 3.4.23.25: saccharopepsin, EC 3.4.23.26: rhodotorulapepsin, EC 3.4.23.28: acrocylindropepsin, EC 3.4.23.29: polyporopepsin, EC 3.4.23.30: pycnoporopepsin, EC 3.4.23.31: scytalidopepsin A, EC 3.4.23.32: scytalidopepsin B, EC 3.4.23.34: cathepsin E, EC 3.4.23.35: barrierpepsin, EC 3.4.23.36: signal peptidase II, EC 3.4.23.38: plasmepsin I, EC 3.4.23.39: plasmepsin II, EC 3.4.23.40: phytepsin, EC 3.4.23.41: yapsin 1, EC 3.4.23.42: thermopsin, EC 3.4.23.43: prepilin peptidase, EC 3.4.23.44: nodavirus endopeptidase, EC 3.4.23.45: memapsin 1, EC 3.4.23.46: memapsin 2, EC 3.4.23.47: HIV- 2 retropepsin, EC 3.4.23.48: plasminogen activator Pla, EC 3.4.23.49: omptin, EC 3.4.23.50: human endogenous retrovirus K endopeptidase, EC 3.4.23.51: Hycl peptidase, and EC 3.4.23.52: preflagellin peptidase.

When used herein the term protein having glutamic protease activity or glutamic protease, are a group of proteolytic enzymes containing a glutamic acid residue within the active site. Such enzyme is described as EC 3.4.23.32 Scytalidopepsin B. Therefore in an embodiment the protein with glutamic protease activity is EC 3.4.23.32 Scytalidopepsin B.

When used herein the term protein having metalloprotease protease activity or metalloprotease, is any protease enzyme whose catalytic mechanism involves a metal. Such enzymes are classified as EC 3.4.24 using the Enzyme Commission number classification. Therefore, the metalloprotease may be selected from: EC 3.4.24.1: atrolysin A, EC 3.4.24.3: microbial collagenase, EC 3.4.24.4: now EC 3.4.24.40 serralysin, EC 3.4.24.6: leucolysin, EC 3.4.24.7: interstitial collagenase, EC 3.4.24.11: neprilysin, EC 3.4.24.12: envelysin, EC 3.4.24. 13: IgA-specific metalloendopeptidase, EC 3.4.24. 14: procollagen N-endopeptidase, EC 3.4.24. 15: thimet oligopeptidase, EC 3.4.24.16: neurolysin, EC 3.4.24.17: stromelysin 1, EC 3.4.24.18: meprin A, EC 3.4.24.19: procollagen C-endopeptidase, EC 3.4.24.20: peptidyl-Lys metalloendopeptidase, EC 3.4.24.21: astacin, EC 3.4.24.22: stromelysin 2, EC 3.4.24.23: matrilysin, EC 3.4.24.24: gelatinase a, EC 3.4.24.25: vibriolysin, EC 3.4.24.26: pseudolysin, EC 3.4.24.27: thermolysin, EC 3.4.24.28: bacillolysin, EC 3.4.24.29: aureolysin, EC 3.4.24.30: coccolysin, EC 3.4.24.31: mycolysin, EC 3.4.24.32: β-lytic metalloendopeptidase, EC 3.4.24.33: peptidyl-Asp metalloendopeptidase, EC 3.4.24.34: neutrophil collagenase, EC 3.4.24.35: gelatinase B, EC 3.4.24.36: leishmanolysin, EC 3.4.24.37: saccharolysin, EC 3.4.24.38: gametolysin, EC 3.4.24.39: deuterolysin, EC 3.4.24.40: serralysin, EC 3.4.24.41: atrolysin B, EC 3.4.24.42: atrolysin C, EC 3.4.24.43: atroxase, EC 3.4.24.44: atrolysin E, EC 3.4.24.45: atrolysin F, EC 3.4.24.46: adamalysin, EC 3.4.24.47: horrilysin, EC 3.4.24.48: ruberlysin, EC 3.4.24.49: bothropasin, EC 3.4.24.50: bothrolysin, EC 3.4.24.51: ophiolysin, EC 3.4.24.52: trimerelysin I, EC 3.4.24.53: trimerelysin II, EC 3.4.24.54: mucrolysin, EC 3.4.24.55: pitrilysin, EC 3.4.24.56: insulysin, EC 3.4.24.57: O-sialoglycoprotein endopeptidase, EC 3.4.24.58: russellysin, EC 3.4.24.59: mitochondrial intermediate peptidase, EC 3.4.24.60: dactylysin, EC 3.4.24.61: nardilysin, EC 3.4.24.62: magnolysin, EC 3.4.24.63: meprin B, EC 3.4.24.64: mitochondrial processing peptidase, EC 3.4.24.65: macrophage elastase, EC 3.4.24.66: choriolysin L, EC 3.4.24.67: choriolysin H, EC 3.4.24.68: tentoxilysin, EC 3.4.24.69: bontoxilysin, EC 3.4.24.70: oligopeptidase A, EC 3.4.24.71: endothelin-converting enzyme 1, EC 3.4.24.72: fibrolase, EC 3.4.24.73: jararhagin, EC 3.4.24.74: fragilysin, EC 3.4.24.75: lysostaphin, EC 3.4.24.76: flavastacin, EC 3.4.24.77: snapalysin, EC 3.4.24.78: gpr endopeptidase, EC 3.4.24.79: pappalysin-1, EC 3.4.24.80: membrane-type matrix metalloproteinase-1, EC 3.4.24.81: ADAM 10 endopeptidase, EC 3.4.24.82: ADAMTS-4 endopeptidase, EC 3.4.24.83: anthrax lethal factor endopeptidase, EC 3.4.24.84: Ste 24 endopeptidase, EC 3.4.24.85: S 2P endopeptidase, EC 3.4.24.86: ADAM 17 endopeptidase, and EC 3.4.24.87: ADAMTS13 endopeptidase.

When used herein the term protein having asparagine peptide lyase activity or asparagine peptide lyase, one of the seven groups in which proteases, also termed proteolytic enzymes, peptidases, or proteinases, are classified according to their catalytic residue. The catalytic mechanism of the asparagine peptide lyases involves an asparagine residue acting as nucleophile to perform a nucleophilic elimination reaction, rather than hydrolysis, to catalyse the breaking of a peptide bond. The asparagine peptide lyase protease may be selected from: EC 3.4.23.44: nodavirus endopeptidase, EC 7.1.2.2: H+-transporting two-sector ATPase, and EC 2.7.7.7: DNA-directed DNA polymerase.

In a particularly preferred embodiment the protein having protease activity is a protein having elastase activity. When used herein, the term elastase or protein having elastase activity refers to a serine protease that is capable of breaking down the extracellular protein elastin, in humans encoded by the ELN gene (ENSG00000049540). It is understood that an elastase or protein having elastase activity may also be able to break down other proteins, such as for example histones.

The inventors herein demonstrate that the proteolytic cleavage and the destruction of cytotoxic activity of Histones by elastase are accelerated by heparin (see Example 1). Therefore, in an embodiment, the protein having serine protease activity is an elastase. In an embodiment the protein having serine protease activity is selected from: neutrophil elastase, chymotrypsin-like elastase family member 1,chymotrypsin-like elastase family member 2A, chymotrypsin-like elastase family member 2B, chymotrypsin-like elastase family member 3A, chymotrypsin-like elastase family member 3B and chymotrypsin C, preferably wherein the protein having elastase activity is neutrophil elastase.

Neutrophil elastase (EC 3.4.21.37, leukocyte elastase, ELANE, ELA 2, elastase 2, neutrophil, elaszym, serine elastase, subtype human leukocyte elastase (HLE)) is a serine proteinase in the same family as chymotrypsin and has broad substrate specificity. The protein is coded in humans by the ELANE gene which is annotated as ENSG00000277571 and ENSG00000197561. Therefore, in an embodiment the neutrophil elastase according to the invention has a protein sequence which is 75%, 80%, 85%, 90%, 91%, 92%, 93%, 94%, 95%, 96%, 97%, 98%, 99% or more or even 100% identical to the protein sequence encoded by the ELANE gene annotated by ENSG00000277571 or ENSG00000197561, or the neutrophil elastase according to the invention has a protein sequence which is encoded by a nucleic acid sequence which is 60%, 65%, 70%, 75%, 80%, 85%, 90%, 91%, 92%, 93%, 94%, 95%, 96%, 97%, 98%, 99% or more or even 100% identical to the nucleic acid sequence of the ELANE gene annotated by ENSG00000277571 or ENSG00000197561.

Chymotrypsin-like elastase family member 1 is also known as pancreatic elastase 1 or EC 3.4.21.36 and is a form of elastase that is produced in the acinar cells of the pancreas, initially produced as an inactive zymogen and later activated in the duodenum by trypsin. The protein is coded in humans by the CELA1 gene which is annotated as ENSG00000139610. Therefore, in an embodiment the neutrophil elastase according to the invention has a protein sequence which is 75%, 80%, 85%, 90%, 91%, 92%, 93%, 94%, 95%, 96%, 97%, 98%, 99% or more or even 100% identical to the protein sequence encoded by the CELA1 gene annotated by ENSG00000139610, or the neutrophil elastase according to the invention has a protein sequence which is encoded by a nucleic acid sequence which is 60%, 65%, 70%, 75%, 80%, 85%, 90%, 91%, 92%, 93%, 94%, 95%, 96%, 97%, 98%, 99% or more or even 100% identical to the nucleic acid sequence of the CELA 1 gene annotated by ENSG00000139610.

Chymotrypsin-like elastase family member 2A (EC 3.4.21.71) is a protein that is coded in humans by the CELA2A gene which is annotated as ENSG00000142615. Therefore, in an embodiment the neutrophil elastase according to the invention has a protein sequence which is 75%, 80%, 85%, 90%, 91%, 92%, 93%, 94%, 95%, 96%, 97%, 98%, 99% or more or even 100% identical to the protein sequence encoded by the CELA2A gene annotated by ENSG00000142615, or the neutrophil elastase according to the invention has a protein sequence which is encoded by a nucleic acid sequence which is 60%, 65%, 70%, 75%, 80%, 85%, 90%, 91%, 92%, 93%, 94%, 95%, 96%, 97%, 98%, 99% or more or even 100% identical to the nucleic acid sequence of the CELA2A gene annotated by ENSG00000142615.

Chymotrypsin-like elastase family member 2B (EC 3.4.21.71) is a protein that is coded in humans by the CELA2B gene which is annotated as ENSG00000215704. Therefore, in an embodiment the neutrophil elastase according to the invention has a protein sequence which is 75%, 80%, 85%, 90%, 91%, 92%, 93%, 94%, 95%, 96%, 97%, 98%, 99% or more or even 100% identical to the protein sequence encoded by the CELA2B gene annotated by ENSG00000215704, or the neutrophil elastase according to the invention has a protein sequence which is encoded by a nucleic acid sequence which is 60%, 65%, 70%, 75%, 80%, 85%, 90%, 91%, 92%, 93%, 94%, 95%, 96%, 97%, 98%, 99% or more or even 100% identical to the nucleic acid sequence of the CELA2B gene annotated by ENSG00000215704.

Chymotrypsin-like elastase family member 3A (EC 3.4.21.70) is a protein that is coded in humans by the CELA3A gene which is annotated as ENSG00000142789. Therefore, in an embodiment the neutrophil elastase according to the invention has a protein sequence which is 75%, 80%, 85%, 90%, 91%, 92%, 93%, 94%, 95%, 96%, 97%, 98%, 99% or more or even 100% identical to the protein sequence encoded by the CELA2A gene annotated by ENSG00000142789, or the neutrophil elastase according to the invention has a protein sequence which is encoded by a nucleic acid sequence which is 60%, 65%, 70%, 75%, 80%, 85%, 90%, 91%, 92%, 93%, 94%, 95%, 96%, 97%, 98%, 99% or more or even 100% identical to the nucleic acid sequence of the CELA2A gene annotated by ENSG00000142789.

Chymotrypsin-like elastase family member 3B (EC 3.4.21.70) is a protein that is coded in humans by the CELA3B gene which is annotated as ENSG00000219073. Therefore, in an embodiment the neutrophil elastase according to the invention has a protein sequence which is 75%, 80%, 85%, 90%, 91%, 92%, 93%, 94%, 95%, 96%, 97%, 98%, 99% or more or even 100% identical to the protein sequence encoded by the CELA3B gene annotated by ENSG00000219073, or the neutrophil elastase according to the invention has a protein sequence which is encoded by a nucleic acid sequence which is 60%, 65%, 70%, 75%, 80%, 85%, 90%, 91%, 92%, 93%, 94%, 95%, 96%, 97%, 98%, 99% or more or even 100% identical to the nucleic acid sequence of the CELA3B gene annotated by ENSG00000219073.

Chymotrypsin C (EC 3.4.21.2) is an enzyme which catalyses cleavage of peptides or proteins. The protein is coded in humans by the chymotrypsin C gene which is also known as CLCR or ELA4, and which is annotated as ENSG00000162438. Therefore, in an embodiment the neutrophil elastase according to the invention has a protein sequence which is 75%, 80%, 85%, 90%, 91%, 92%, 93%, 94%, 95%, 96%, 97%, 98%, 99% or more or even 100% identical to the protein sequence encoded by the chymotrypsin C gene annotated by ENSG00000162438, or the neutrophil elastase according to the invention has a protein sequence which is encoded by a nucleic acid sequence which is 60%, 65%, 70%, 75%, 80%, 85%, 90%, 91%, 92%, 93%, 94%, 95%, 96%, 97%, 98%, 99% or more or even 100% identical to the nucleic acid sequence of the chymotrypsin C gene annotated by ENSG00000162438.

The inventors herein further demonstrate that the proteolytic cleavage and the destruction of cytotoxic activity of Histones by (activated) protein C are accelerated by heparin (see Example 1). Therefore, in an embodiment, the protein having protease activity is Protein C. In a preferred embodiment, the protein having protease activity is Activated Protein C (APC). Protein C (EC 3.4.21.69) is also known as autoprothrombin IIA and blood coagulation factor XIX, is a zymogen, that is, an inactive enzyme. The activated form plays an important role in regulating anticoagulation, inflammation, and cell death and maintaining the permeability of blood vessel walls in humans and other animals. Activated protein C (APC) performs these operations primarily by proteolytically inactivating proteins Factor Va and Factor Villa. APC is classified as a serine protease since it contains a residue of serine in its active site. The protein is coded in humans by the PROC gene, and which is annotated as ENSG00000115718. Therefore, in an embodiment the neutrophil elastase according to the invention has a protein sequence which is 75%, 80%, 85%, 90%, 91%, 92%, 93%, 94%, 95%, 96%, 97%, 98%, 99% or more or even 100% identical to the protein sequence encoded by the PROC gene annotated by ENSG00000115718, or the neutrophil elastase according to the invention has a protein sequence which is encoded by a nucleic acid sequence which is 60%, 65%, 70%, 75%, 80%, 85%, 90%, 91%, 92%, 93%, 94%, 95%, 96%, 97%, 98%, 99% or more or even 100% identical to the nucleic acid sequence of the PROC gene annotated by ENSG00000115718.

When used herein the term negatively charged polysaccharide refers to a polymer of saccharides comprising or consisting of negatively charged saccharides. Preferably the negatively charged polysaccharide is or comprises a glycosaminoglycan, meaning the negatively charged polysaccharide comprises repeating disaccharide units. When used herein, glycosaminoglycans (GAGs), also known as mucopolysaccharides, are long linear polysaccharides consisting of repeating disaccharide units (i.e. two-sugar units). The repeating two-sugar unit consists of a uronic sugar and an amino sugar, with the exception of keratan, where in the place of the uronic sugar it has galactose. Therefore, the repeating disaccharide units consist or comprise preferably of a uronic sugar and an amino sugar or galactose and an amino sugar. Uronic sugars (also referred to as uronic acid) are sugars in which the hydroxyl group furthest from the carbonyl group has been oxidized to a carboxylic acid. Non-limiting examples of uronic sugars are glucuronic acid, gluconic acid, iduronic acid and isosaccharinic acid. An amino sugar when used herein is a sugar molecule in which a hydroxyl group has been replaced with an amine group. Non-limiting examples are glucosamine, N-acetylglucosamine, galactosamine, L-daunosamine, and sialic acid. Therefore, in an embodiment the negatively charged polysaccharide is or comprises a glycosaminoglycan (GAG).

The polysaccharide according to the invention is negatively charged. Preferably the polysaccharide is negatively charged because it contains variably sulphated repeating disaccharide units. For example iduronic acid may be sulphated at the 2-O position to give 2-O-sulfated iduronic acid, or glucosamine may be sulfated at the 6-O and/or the N position to give 6-O-sulfated, N-sulfated glucosamine. Other sulfated saccharides are known to the skilled person and may be incorporated in the negatively charged polysaccharide of the invention. Therefore, in an embodiment the negatively charged polysaccharide consists of or comprises a variably sulphated repeating disaccharide unit.

Therefore, when used herein the negatively charged polysaccharide has at least 10%, for example 20%, 30%, 40%, 50%, 60%, 70%, 80%, 85%, 90%, 95% or more such as 98%, 99% or even 100% disaccharides selected from GlcA-GlcNAc, GlcA-GlcNS, IdoA-GlcNS, IdoA(2S)-GlcNS, IdoA-GlcNS(6S), IdoA(2S)-GlcNS(6S), wherein GlcA=β-D-glucuronic acid, IdoA=α-L-iduronic acid, IdoA(2S)=2-O-sulfo-α-L-iduronic acid, GlcNAc=2-deoxy-2-acetamido-α-D-glucopyranosyl, GlcNS=2-deoxy-2-sulfamido-α-D-glucopyranosyl, GlcNS(6S)=2-deoxy-2-sulfamido-a-D-glucopyranosyl-6-O-sulfate.

Ideally the negatively charged polysaccharide has a molecular weight of at least 2 kDa, preferably at least 3 kDa, more preferably at least 4 kDa. Therefore, the negatively charged polysaccharide has a chain length of at least 10, preferably at least 15 more preferably at least 20 saccharide units, or at least 5, more preferably at least 7 most preferably at least 10 disaccharide units. It is understood that the negatively charged polysaccharide may be a mixture of distinct but related polymers, in which case the molecular weight or chain size refers to the average molecular weight or chain size. The average molecular weight is determined by summing the weights of all the chains and then dividing by the total number of chains. The average chains length is determined by summing the lengths of all the chains and then dividing by the total number of chains. In an embodiment the negatively charged polysaccharide has a molecular weight of at most 50 kDa, preferably at most 40 kDa, more preferably at most 30 kDa. Therefore, the negatively charged polysaccharide has a chain length of at most 250, preferably at most 200 more preferably at most 150 saccharide units, or at most 125, more preferably at most 100 most preferably at most 75 disaccharide units.

In an embodiment the negatively charged polysaccharide is heparin. When used herein, heparin refers to a polymer with a molecular weight generally ranging from 3 to 30 kDa, which is a member of the glycosaminoglycan family of carbohydrates and also includes the closely related molecule heparan sulfate. Heparin comprises variably sulfated repeating disaccharide units, the main disaccharide units that occur in heparin are GlcA-GlcNAc, GlcA-GlcNS, IdoA-GlcNS, IdoA(2S)-GlcNS, IdoA-GlcNS(6S), and IdoA(2S)-GlcNS(6S). The most common disaccharide unit in heparin is composed of a 2-O-sulfated iduronic acid and 6-O-sulfated, N-sulfated glucosamine, IdoA(2S)-GlcNS(6S). Therefore, in an embodiment the negatively charged polysaccharide is heparin.

It was further found by the inventors that in an especially beneficial embodiment of the invention, a low-or non-anticoagulant heparin can be used. It was found that heparin has both anticoagulant and anti-inflammatory functions, and that most of the anti-inflammatory pharmacology of heparin is unrelated to anticoagulant activity (3). Therefore, in an embodiment the negatively charged polysaccharide is a low anticoagulant heparin or non-anticoagulant heparin. Low or non-anticoagulant heparin are known to the skilled person, and refer to specific heparin fraction or modified heparin that have reduced or no anti-coagulant action. When used herein the term heparin thus encompasses “regular” heparin and low- or non-antiocoagulant heparin. With heparin is meant naturally occurring heparin or synthetic heparin that has a clear anticoagulant function. With regular heparin is meant either naturally occurring or synthetic heparin having both anticoagulant and anti-inflammatory actions. An example of regular heparin is unfractionated heparin (UFH).

For example pentasaccharide-depleted heparin may be used as a low- or non-anticoagulant heparin, and has been described for example in EP2731616A1 which is incorporated by reference in its entirety. The term pentasaccharide-depleted in this context is used to refer to a fraction of heparin wherein the content of pentasaccharides is substantially reduced in comparison to commercially available heparin. A non-limiting example of pentasaccharide-depleted heparin is M6229 (see e.g. https://clinicaltrials.gov/ct2/show/NCT05208112).

Other non-limiting examples of low or non-anticoagulant heparin include docipartstat, chemically O-desulfated heparin, regioselectively desulfated heparins and ‘glycol-split’ heparins. Docipartstat is a glycosaminoglycan derived from porcine heparin and also known as DSTAT, CX-01, 2-O,3-O desulfated heparin or ODSH. Docipartstat is currently tested in combination with standard chemotherapy for the treatment of Acute Myeloid Leukemia (AML), see e.g. https://clinicaltrials.gov/ct2/show/NCT04571645. Regioselectively desulfated heparins are heparins with partial remove sulfate groups and have been described in Takano et al. (4), which is hereby incorporated by reference in its entirety. Chemically O-desulfated heparin includes for example partial or completely 6-O-desulfated heparin and have been described in Kariya et al. (5), hereby incorporated by reference in its entirety. Glycol-split heparin refers to heparin and low-molecular-weight heparins (LMWHs) like sevuparin which has been subjected to periodate oxidation followed by borohydride reduction. The process converts the well-known antithrombotics into their glycol-split (gs) derivatives of the reduced oxyheparin (RO) type, some of which are currently being developed as potential anti-cancer and anti-inflammatory drugs, and have been described in Alekseeva et al. (6), which is hereby incorporated by reference in its entirety. Therefore, in an embodiment, the negatively charged polysaccharide is selected from pentasaccharide-depleted heparin, dociparstat, chemically O-desulfated heparin, sevuparin, regioselectively desulfated heparins and glycol-split heparins.

The present invention relates to a complex of a negatively charged polysaccharide and a protein having protease activity. The term complex when used herein should be interpreted as an association of the individual components. Therefore, the term may for example refer to binding of the individual components (the negatively charged polysaccharide and the protein having protease activity) by one or more selected from: covalent binding, van der Waals forces binding, electrostatic force binding, hydrogen bridge binding, or ionic force binding. Therefore, in an embodiment bound by van der Waals forces, bound by electrostatic forces, bound by hydrogen bridges, or bound by ionic forces. Therefore, in an embodiment, the negatively charged polysaccharide and the protein having protease activity are covalently bound, bound by van der Waals forces, bound by electrostatic forces, bound by hydrogen bridges, bound by ionic forces or bound by a combination of two or more of these. One option for covalent coupling is provided by the terminal serine group in heparin which can be used to create an amide bond with the proteolytic moiety.

The complex may be formed using electrostatic interactions between the negatively charged polysaccharide and the protease. For example, such interactions may be mediated by a recognition motif, such as the Cardin-Weintraub consensus sequence which is also found in NE (see e.g. (7) and (8)), however the interaction does not necessarily need to be mediated by such sequence.

Therefore in an embodiment the complex is obtained or obtainable by bringing together the negatively charged polysaccharide and the protein having protease activity. For example the negatively charged polysaccharide and the protein having protease activity can be brought together in approximately equimolar ratios, or a small excess of negatively charged polysaccharide can be used. Therefore the invention further relates to a method of obtaining the complex described herein comprising bringing together a negatively charged polysaccharide and a protein having protease activity. In a preferred embodiment the molar ratio of the negatively charged polysaccharide to the protein having protease activity is approximately 1:1, for example 1:2, 1:1 or 2:1, or a small excess of negatively charged polysaccharide can be used, for example 2:1, 3:1, 4:1, 5:1, 6:1, 7:1, 8:1, 9:1 or even 10:1. Preferably the negatively charged polysaccharide and the protein having protease activity are brought together in a suitable solvent, such as an aqueous buffer or saline solution. For example, the negatively charged polysaccharide and the protein having protease activity can be brought together by adding a solution comprising a negatively charged polysaccharide to a solution comprising the protein having protease activity or vice versa. Alternatively the negatively charged polysaccharide and the protein having protease activity can be brought together by dissolving a negatively charged polysaccharide in a solution comprising the protein having protease activity or vice versa.

It is further envisioned that a complex of negatively charged polysaccharide and a protein having protease activity wherein the complex is formed through a covalent bond may be advantageous as it prevents dissociation of the negatively charged polysaccharide and the protein having protease activity. It is envisioned that such complex may even further lower the needed amount of negatively charged polysaccharide as any excess (non-bound negatively charged polysaccharide) can be removed. Method for attaching the negatively charged polysaccharide and the protein having protease activity to each other are known to the skilled person, for example covalent coupling can be achieved using the free amine or carboxyl group of the terminal serine group in heparin which can be used to create an amide bond with a proteolytic moiety.

In a second aspect the invention relates to a pharmaceutical composition comprising the complex according to the first aspect of the invention and a pharmaceutically acceptable carrier. Pharmaceutically acceptable carriers are known to the skilled person and may for example be a salt solution such a physiological salt solution or a buffer solution. The skilled person is however aware of other suitable pharmaceutically acceptable carriers or to determine a pharmaceutically acceptable carrier suitable for the intended application of the complex as described herein.

It is envisioned that the complex as described herein finds use in a medical treatment. Therefore, in a third aspect the invention relates to the complex according to the first aspect of the invention or the pharmaceutical composition according to the second aspect of the invention for use as a medicament. Alternatively, the invention relates to a method of treatment, comprising administering the complex according to the first aspect of the invention or the pharmaceutical composition according to the second aspect of the invention to a subject in need thereof.

### Extracellular Histones

Histones are nuclear proteins and core histones H2A, H2B, H3 and H4, together with linker histone H1, organize DNA into chromatin and regulate gene expression. The pathologic release of extracellular histones is known to have clinical consequences as these proteins are cytotoxic towards host tissues and serve as damage-associated molecular patterns (DAMPs). Extracellular histones have been associated with Immunothrombosis related disorders such as sepsis or ischemia-reperfusion injury (IRI) in kidneys but also trauma.

The inventors and others have already demonstrated the negative role of eHs, for example in immunothrombosis. Therefore, the results provided here in examples 1 and 2 render it plausible that the complex as described herein may be used to treat, prevent or ameliorate a disease or disorder mediated by extracellular histones. This is rendered plausible because the complex may assist in the degradation of eHs (in the extracellular space), while when using such complex, the amount of heparin needed is much lower as when only using heparin, thereby counteracting potential issues related to the anticoagulant action of heparin and the development of a self-enforcing cascade of histone release leading to serious tissue damage. Therefore, in a fourth aspect the invention relates to the complex according to the first aspect of the invention or the pharmaceutical composition according to the second aspect of the invention for use in the treatment, prevention or amelioration of an extracellular histone-mediated disease or disorder. In an alternative embodiment the invention relates to a method of treating, preventing, or ameliorating a subject suffering from an extracellular histone-mediated disease or disorder, comprising administering the complex according to the first aspect of the invention or the pharmaceutical composition according to the second aspect of the invention to the subject. When used herein, the term extracellular histone-mediated disease or disorder refers to any disease, disorder or event where histones are released extracellularly to exert a cytotoxic effect. Examples of extracellular histone-mediated diseases or disorders are immunothrombosis related diseases or disorders (as defined herein below), and trauma.

### Immunothrombosis

Immunothrombosis is a term referring to complex responses of the human body to sterile and non-sterile inflammatory stimuli. The complex responses arise from interactions between an activated hemostatic system, an activated immune system and an activated complement system. The activated systems can involve platelet activation and coagulation (hemostatic system), neutrophils activation and neutrophil extracellular trap (NET) formation (immune system), and C5a formation and C5b-9 complex formation (complement system). The interaction between the systems contain amplifying loops that can boost the immunothrombotic response and enhance the risk of aggravating morbidity and mortality. eHs constitute a molecular part of the mechanisms by which the amplifying loops operate. Therefore, when used herein the term immunothrombosis related disease or disorder should be interpreted as any disease or disorder where one or more mechanisms of immunothrombosis occurs. Preferably the disease or disorder is characterized that at least one of the symptoms of the disease or disorder arises from immunothrombosis.

Therefore, in an embodiment the invention relates to the complex according to the first aspect of the invention or the pharmaceutical composition according to the second aspect of the invention for use in the treatment, prevention or amelioration of an immunothrombosis related disease or disorder. In an alternative embodiment the invention relates to a method of treating, preventing, or ameliorating a subject suffering from an immunothrombosis related disease or disorder, comprising administering the complex according to the first aspect of the invention or the pharmaceutical composition according to the second aspect of the invention to the subject.

Non-limiting examples of immunothrombosis related diseases or disorders are sepsis, inflammation, cancer, burn wounds, severe trauma, stroke, ischaemic reperfusion, acute myocardial infarct, organ transplantation, Anti-Neutrophilic Cytoplasmic Autoantibodies (ANCA) associated vasculitis, thrombotic microangiopathy, acute respiratory distress syndrome, thrombotic thrombocytopenia purpura, endotoxemia, pancreatitis, peritonitis, and thromboembolic disease. Therefore, in an embodiment, the immunothrombosis related disease or disorder is selected from: sepsis, inflammation, cancer, burn wounds, severe trauma, stroke, ischaemic reperfusion, acute myocardial infarct, organ transplantation, Anti-Neutrophilic Cytoplasmic Autoantibodies (ANCA) associated vasculitis, thrombotic microangiopathy, acute respiratory distress syndrome, thrombotic thrombocytopenia purpura, endotoxemia, pancreatitis, peritonitis, thromboembolic disease.

### REFERENCES

1. Redini F, Tixier J M, Petitou M, Choay J, Robert L, Hornebeck W. Inhibition of leucocyte elastase by heparin and its derivatives. Biochem J 1988; 252(02):515-519
2. Nicolaes GAF, Sørensen KW, Friedrich U, et al. Altered inactivation pathway of factor Va by activated protein C in the presence of heparin. Eur J Biochem 2004; 271(13):2724-2736
3 Rao et al. Am J Physiol Cell Physiol. 2010 July; 299(1):C97-110.
4. Takano et al., Japanese Journal of Thrombosis and Hemostasis. 10. 45-55.
5. Kariya et al. J Biol Chem. 2000 Aug. 25; 275(34):25949-58.
6. Alekseeva et al. Anal Bioanal Chem. 2014 January; 406(1):249-65.
7. Gandhi et al. Chem Biol Drug Des 2008; 72:455-482
8. Muñoz et al. Arterioscler Thromb Vasc Biol. 2004 September; 24(9): 1549-1557.
9. Ohnishi et al. Chem. Pharm. Bull. 33(2) 674-678 (1985)
10. Chen et al. Carbohydr Polym. 2017 Feb. 10; 157:244-250.

### EXAMPLES

### Example 1

The proteolytic cleavage and the destruction of cytotoxic activity of Histones by elastase are accelerated by unfractionated heparin (UFH) and low anticoagulant heparin (M6229).

### Materials & Methods

Histones H2B, H3 and H4 were purchased from Roche or NEB. Native human Neutrophil Elastase (NE) (ab91099) was purchased from Abcam.

### Cleavage of histone isoforms

To evaluate the potential of NE to cleave histones, 5 nM of NE was added to 5 μg/ml histone H2B, H3 and H4 in HN buffer (25 mM HEPES, 140 mM NaCl, 5 mM CaCl2, pH 7.4) and incubated at 37oC. Samples were taken at several time points and the reaction was stopped by addition of a reaction volume to Laemmli sample buffer. Samples were next incubated for 5 minutes at 95° C. The samples were run on an SDS-PAGE gel and analyzed by Western blotting using antibodies against H2B, H3 and H4. To test the effects of heparins, heparins were complexed with NE prior to addition to the reaction mixture. Complexation of NE with heparins reduced the NE-induced conversion of a small chromogenic substrate (not shown).

### Histone Western Blotting

Histone fragmentation was determined using a semi-quantitative method. Briefly, samples were subjected to SDS-PAGE gel electrophoresis and transferred to PVDF membranes (Bio-Rad Laboratories) using semi-dry blotting. Membranes were blocked and incubated overnight at 4° C. with a primary specific histone antibody: mouse monoclonal anti-H2B (SC-515808, Bio-connect), rabbit polyclonal anti-histone H3 (ab94817, Abcam), and mouse monoclonal anti-H4 (L64C1, Cell signaling). This was followed by a secondary biotin-conjugated donkey anti-rabbit IgG (ab97083, Abcam) for Histone H3 or a HRP-conjugated goat anti-mouse (p0477, Dako) for histone H2B or H4 for 1 hour minutes at RT. For histone H3 it was incubated for 30 minutes with a streptavidin-biotin/HRP complex (Vectastain) at RT. Histone bands were detected by luminescent ECL substrate (Advansta). Resulting band densities were quantified by ImageQuant TL software (GE Healtcare, Little Chalfont, UK).

### Detection of Histone Cytotoxicity

EA.hy926 cells were plated on a 24-well plate, and grown to 80% confluency in complete DMEM medium (Thermofisher Scientific) supplemented with HAT (hypoxanthine-aminopterin-thymidine, Gibco). After washing two times with phosphate buffered saline (PBS, Gibco). After washing two times with PBS, cells were incubated with 2.7 nM Histone H3 and various amounts of UFH or M6229 in the presence or absence of 5 nM NE in DMEM without Fetal Bovine Serum. After 1 hour of incubation at 37oC, the medium containing detached cells was collected, and the attached cells were collected using 0.05% trypsin-EDTA (Gibco). Collected cells were pelleted and reconstituted in binding buffer (10 mM HEPES, 150 mM NaCl, 5 mM KCl, 2 mM MgCl2 and 3.3 mM CaCl2) containing 2.5 μg/ml Propidium lodide (Sigma). After a 10 minutes incubation in the dark, the percentage of PI positive cells was measured using flow cytometry with BD Accuri C6 and analyzed using the BD CFlow plus software.

### Results

### NE Cleaves Histones

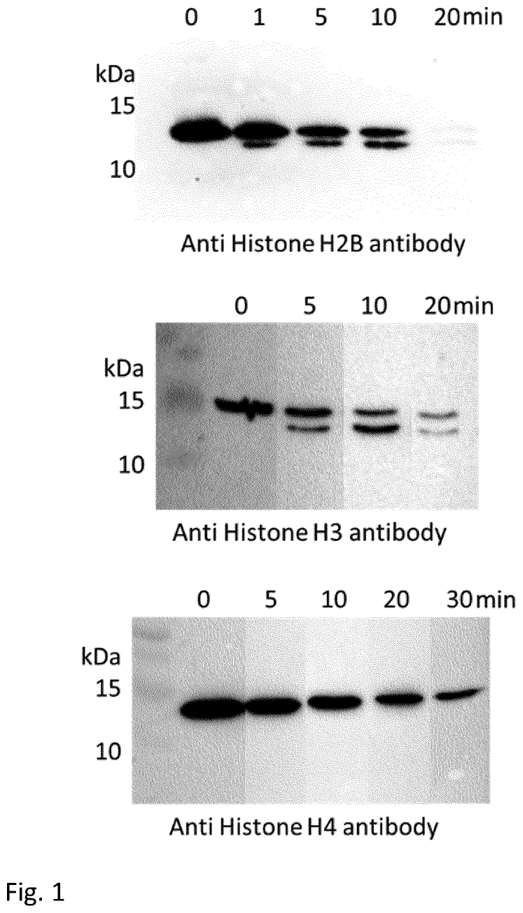

It was shown that NE cleaves Histones H2B, H3 and H4 in a time dependent manner. FIG. 1 illustrates the proteolytic cleavage of Histones by NE. Histone H4 is cleaved into smaller fragments that are not visualized by Western blotting using the monoclonal antibody employed here.

### NE-Catalyzed Cleavage of Histones is Accelerated by UFH

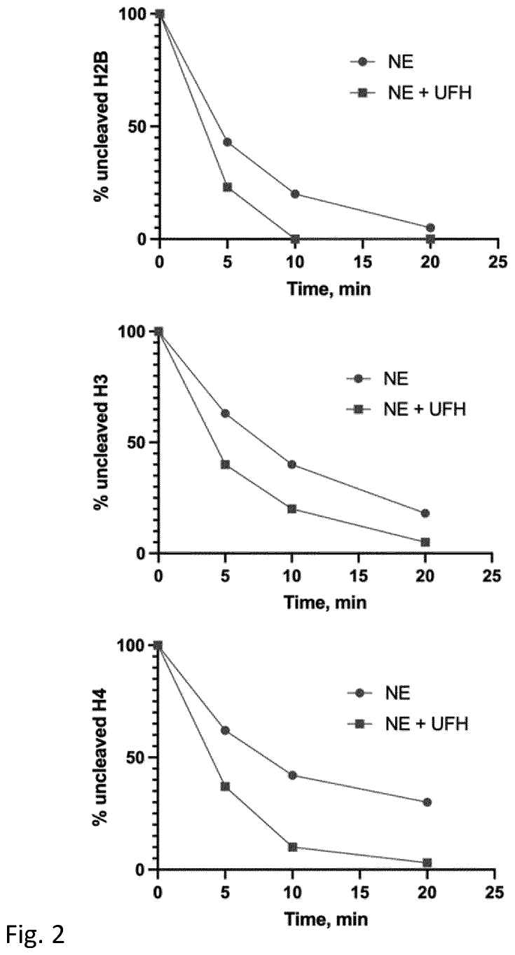

Complexing NE with UFH accelerated the NE-catalyzed degradation of Histones H2B, H3 and H4. The time courses of NE-induced Histone degradation for the free NE or NE-UFH complex are shown in FIG. 2.

### NE enhances the cytoprotective effects of UFH and M6229

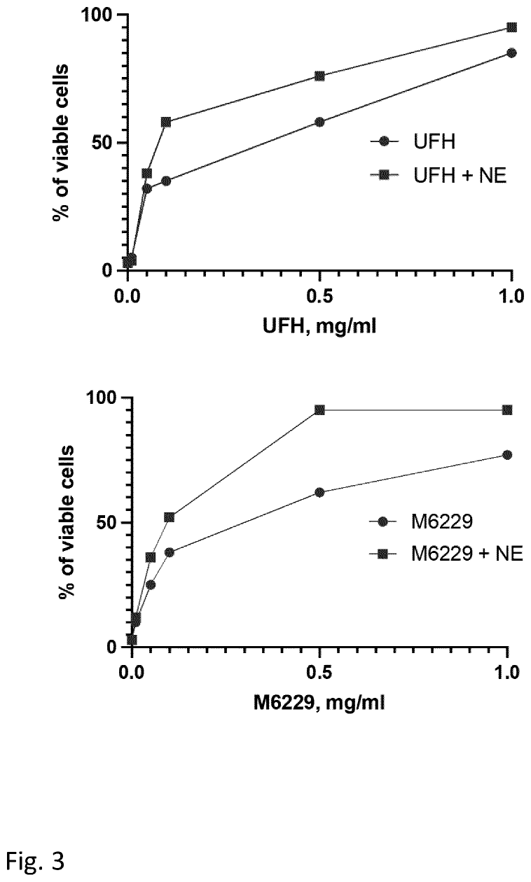

When endothelial cells (EA.hy926) are incubated with Histone H3 they lose viability due to the cytotoxic effects of H3. Incubation of H3 with heparins such as UFH and M6229 neutralizes the cytotoxic effects of H3 in a dose dependent manner (FIG. 3). Complexation of UFH and M6229 with NE enhances the cytoprotective effects of UFH and M6229 (FIG. 3). NE had by itself no effect on the viability of EA.hy926 cells (not shown).

### Example 2

The proteolytic cleavage and the destruction of cytotoxic activity of Histones by activated protein C are accelerated by unfractionated heparin (UFH).

### Materials & Methods

Histone H3 was purchased from Roche. Human activated protein C (APC) was produced by recombinant techniques.

### Cleavage of Histone Isoforms

To evaluate the potential of APC to cleave histones, 10 nM of NE was added to 5 μg/ml histone H3 in HN buffer (25 mM HEPES, 140 mM NaCl, 5 mM CaCl2, pH 7.4) and incubated at 37° C. Samples were taken at several time points and the reaction was stopped by addition of a reaction volume to Laemmli sample buffer. Samples were next incubated for 5 minutes at 95° C. and on an SDS-PAGE gel and analyzed by Western blotting using antibodies against H3. To test the effects of heparins, heparin was complexed with APC prior to addition to the reaction mixture.

### Histone Western Blotting

Histone H3 fragmentation was determined using a semi-quantitative method. Briefly, samples were subjected to SDS-PAGE gel electrophoresis and transferred to PVDF membranes (Bio-Rad Laboratories) using semi-dry blotting. Membranes were blocked and incubated overnight at 4° C. with a primary specific rabbit polyclonal anti-histone H3 (ab94817, Abcam). This was followed by a secondary biotin-conjugated donkey anti-rabbit IgG (ab97083, Abcam) and a streptavidin-biotin/HRP complex (Vectastain) incubation at RT. Histone bands were detected by luminescent ECL substrate (Advansta). Resulting band densities were quantified by ImageQuant TL software (GE Healtcare, Little Chalfont, UK).

### Detection of Histone Cytotoxicity

EA.hy926 cells were plated on a 24-well plate, and grown to 80% confluency in complete DMEM medium (Thermofisher Scientific) supplemented with (hypoxanthine-aminopterin-thymidine, Gibco). After washing two times with phosphate buffered saline (PBS, Gibco), cells were incubated with 2.7 nM H3 with various amounts of UFH in the presence or absence of 10 nM APC in DMEM without Fetal Bovine Serum. After 1 hour incubation at 37oC, the medium containing detached cells was collected, and the attached cells were collected using 0.05% trypsin-EDTA (Gibco). Collected cells were pelleted and reconstituted in binding buffer (10 mM HEPES, 150 mM NaCl, 5 mM KCl, 2 mM MgCl2 and 3.3 mM CaCl2) containing 2.5 μg/ml Propidium lodide (Sigma). After a 10 minutes incubation in the dark, the percentage of PI positive cells was measured using flow cytometry with BD Accuri C6 and analyzed using the BD CFlow plus software.

### Results

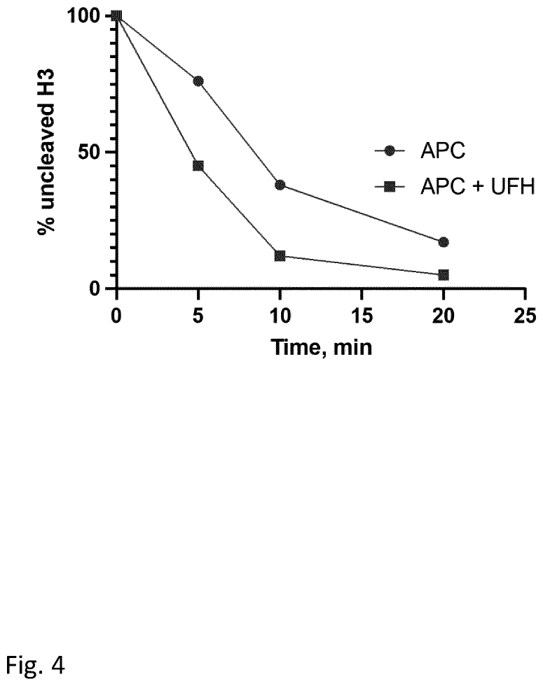

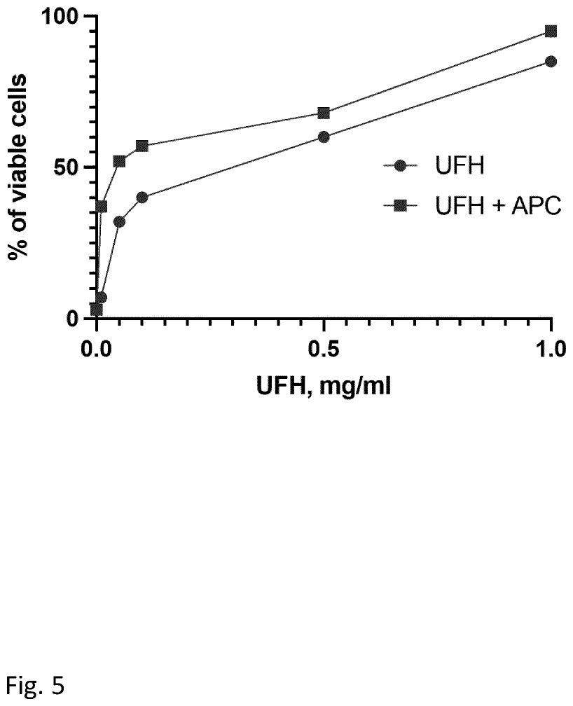

APC cleaves histones and heparins accelerate the APC-catalyzed cleavage APC cleaves Histone H3 in a time-dependent manner (FIG. 4). Complexing APC with UFH accelerated the APC-catalyzed degradation of Histone H3 as shown in FIG. 5.

### APC Enhances the Cytoprotective Effects of UFH

When endothelial cells (EA.hy926) are incubated with Histone H3 they lose viability due to the cytotoxic effects of H3. Incubation of H3 with heparins neutralizes the cytotoxic effects of Histone H3 in a dose dependent manner (FIG. 5). Complexation of UFH with APC enhances the cytoprotective effects of UFH (FIG. 5). APC had by itself no effect on the viability of EA.hy926 cells (not shown).

1. A complex of a negatively charged polysaccharide and a protein having protease activity for use in the treatment, prevention or amelioration of an extracellular histone-mediated disease or disorder,
wherein the protein having protease activity is a serine protease, a cysteine protease, a threonine protease, an aspartic protease, a glutamic protease, a metalloprotease, or an asparagine peptide lyase, and
wherein the negatively charged polysaccharide is a glycosaminoglycan (GAG).

2. The complex for use according to claim 1, wherein the protein having protease activity is an elastase.

3. The complex for use according to claim 1, wherein the protein having protease activity is Protein C.

4. The complex for use according to claim 1, wherein the negatively charged polysaccharide consists of or comprises a variably sulphated repeating disaccharide unit.

5. The complex for use according to claim 1, wherein the negatively charged polysaccharide is heparin.

6. The complex for use according to claim 1, wherein the negatively charged polysaccharide is a low anticoagulant heparin or non-anticoagulant heparin.

7. The complex for use according to claim 1, wherein the negatively charged polysaccharide is selected from pentasaccharide-depleted heparin, dociparstat, chemically O-desulfated heparin, sevuparin, regioselectively desulfated heparins and ‘glycol-split’ heparins.

8. The complex for use according to claim 1, wherein the negatively charged polysaccharide and the protein having protease activity are covalently bound, bound by van der Waals forces, bound by electrostatic forces, bound by hydrogen bridges, bound by ionic forces, or bound by a combination of two or more of these.

9. The complex for use according to claim 1, wherein the complex is comprised in a pharmaceutical composition.

10. The complex for use according to claim 1, wherein the extracellular histone-mediated disease or disorder is an immunothrombosis related disease or disorder, or trauma.

11. The complex for use according to claim 10, wherein the immunothrombosis related disease or disorder is selected from: sepsis, inflammation, cancer, burn wounds, severe trauma, stroke, ischaemic reperfusion, acute myocardial infarct, organ transplantation, Anti-Neutrophilic Cytoplasmic Autoantibodies (ANCA) associated vasculitis, thrombotic microangiopathy, acute respiratory distress syndrome, thrombotic thrombocytopenia purpura, endotoxemia, pancreatitis, peritonitis, thromboembolic disease.

12. A complex of a negatively charged polysaccharide and a protein having protease activity,
wherein the protein having protease activity is a serine protease, a cysteine protease, a threonine protease, an aspartic protease, a glutamic protease, a metalloprotease, or an asparagine peptide lyase, and
wherein the negatively charged polysaccharide is a glycosaminoglycan (GAG); and
wherein the negatively charged polysaccharide and the protein having protease activity are covalently bound.

13. The complex for use according to claim 2, wherein the elastase is selected from: neutrophil elastase, chymotrypsin-like elastase family member 1, chymotrypsin-like elastase family member 2A, chymotrypsin-like elastase family member 2B, chymotrypsin-like elastase family member 3A, chymotrypsin-like elastase family member 3B and chymotrypsin C.

14. The complex for use according to claim 13, wherein the protein having elastase activity is neutrophil elastase.

15. The complex according to claim 3, wherein the protein having protease activity is Activated Protein C (APC).

16. The complex according to claim 9, wherein the complex further comprises a pharmaceutically acceptable carrier
